(19) United States
(12) Patent Application Publication
(10) Pub. No.: US 2025/0377360 A1
(43) Pub. Date: Dec. 11, 2025

(54) METHODS FOR DETECTING A CANCER

(71) Applicant: Georgia Tech Research Corporation, Atlanta, GA (US)

(72) Inventors: Ashkan Zandi, Lawrenceville, GA (US); Nasrin Hooshmand, Atlanta, GA (US)

(21) Appl. No.: 19/231,083

(22) Filed: Jun. 6, 2025

Related U.S. Application Data
(60) Provisional application No. 63/657,531, filed on Jun. 7, 2024.

(51) Int. Cl. G01N 33/574; G01N 1/38; G01N 33/543

(52) U.S. Cl. CPC G01N 33/57438; G01N 1/38; G01N 33/5438; G01N 33/57492; G01N 2333/70503; G01N 2333/70564; G01N 2333/70585; G01N 2333/70596

(57) ABSTRACT

The present disclosure is directed towards methods for evaluating the presence of a cancer within a subject. Such methods include obtaining a stool sample from the subject, identifying a biological component in the stool sample, wherein the identity of the biological component is indicative of the presence or absence of a cancer. The methods disclosed herein may further include quantifying the amount of the biological component in the stool sample, wherein the quantity of the biological component is indicative of the presence or absence of a cancer. The present disclosure is also directed to devices for evaluating the presence of cancer within a subject, such device include a biological sample pre-conditioner configured to prepare a biological sample for quantification of a biological component in the biological sample, and a biological component detector, configured to identify, and optionally quantify the amount of, the biological component in the biological sample.

### CROSS-REFERENCE TO RELATED APPLICATION

This application claims the benefit of U.S. Provisional Application No. 63/657,531 filed on Jun. 7, 2024, the entire contents of which are incorporated by reference herein.

### BACKGROUND OF THE DISCLOSURE

Pancreatic cancer, often referred to as pancreatic adenocarcinoma, is a relatively rare but highly aggressive form of cancer that affects the pancreas. It is characterized by the uncontrolled growth of cells in the pancreas and can be challenging to detect in its early stages. Pancreatic cancer is often diagnosed at an advanced stage, which makes it one of the deadliest forms of cancer. Pancreatic cancer has two main types. The first main type is Exocrine Pancreatic Cancer which is the most common type of pancreatic cancer, accounting for about 95% of cases. It begins in exocrine cells of the pancreas, which are responsible for producing digestive enzymes. Adenocarcinoma is the most common subtype of exocrine pancreatic cancer. The second main type is Endocrine Pancreatic Cancer (Pancreatic Neuroendocrine Tumors, or “PNETs”). PNETs are less common and develop in the endocrine cells of the pancreas, responsible for producing hormones like insulin and glucagon. PNETs are typically slower growing than exocrine tumors.

FIG. 1A is an anatomical illustration of the pancreas. This schematic depicts the location and depth of the pancreas within the human body, emphasizing its relative position behind the stomach and in close proximity to other vital organs such as the small intestine, spleen, liver, and major blood vessels. The illustration highlights the pancreas' deep-seated placement, underscoring the complexities and challenges associated with accessing the organ for diagnostic and therapeutic procedures. All sizes have been exaggerated for demonstration purposes. The inset of FIG. 1A provides a focused schematic of the pancreas, gallbladder, and common bile duct, illustrating the pathway of digestive enzymes from the pancreas to the duodenum. It highlights the integral role of the pancreas in digestion, showcasing the release and flow of enzymes essential for the breakdown of food in the gastrointestinal tract.

FIG. 1B is a schematic representation that provides a comprehensive view of the pancreas afflicted by a pancreatic tumor, highlighting the involvement of adjacent lymph nodes and the metastasis to the liver. FIG. 1B details the pancreatic secretion within the duodenum, including biomarkers CA19-9 and CEA, which may serve as indicators of pancreatic cancer. Additionally, the diagram underscores the presence of pancreatic cancer-derived exosomes.

Detecting pancreatic cancer in its early stages is challenging because it often does not cause noticeable symptoms until it has reached an advanced stage. However, there are some methods used for early detection and screening, although they have limitations. Imaging methods including abdominal ultrasound, computed tomography (CT) scans, magnetic resonance imaging (MRI), and endoscopic ultrasound (EUS) can be used. Those imaging techniques can help identify pancreatic tumors, but they are not always effective in detecting small or early-stage tumors. Blood biochemistry tests can also be used for certain blood markers, such as CA19-9 and CEA, which can be elevated in people with pancreatic cancer. However, those blood tests can be invasive to the patient. To date, various other techniques have also been used to measure protein levels in a solution including enzyme-linked immunosorbent assay (ELISA), cyclic voltammetry (CV), electrochemical impedance spectroscopy (EIS) and differential pulse voltammetry (DPV). Such techniques, have also been found deficient for early detection of pancreatic cancer.

There are challenges and limitations in the early diagnosis of pancreatic cancer, which is the main reason for pancreatic cancer having one of the highest mortality rates among all cancers. Pancreatic cancer is often diagnosed at an advanced stage when it has already spread to nearby organs or distant sites, making it more difficult to treat. Late detection in the context of pancreatic cancer refers to the fact that many cases of pancreatic cancer are not diagnosed until the disease has reached an advanced stage. By the time pancreatic cancer is discovered, it has often spread beyond the pancreas to nearby organs or even to distant parts of the body through metastasis. Late detection is a significant issue in pancreatic cancer due to its aggressive nature. It tends to grow and spread quickly compared to some other types of cancer. As a result, when it is diagnosed at an advanced stage, it is often in an advanced state of progression. Early detection of pancreatic cancer is difficult as the disease is asymptomatic in its early stages. When symptoms do appear, they are often vague and can be mistaken for other common conditions. For example, symptoms like abdominal pain, weight loss, jaundice (yellowing of the skin and eyes), and digestive issues can be indicative of, but are not specific to, pancreatic cancer. The lack of specific early symptoms contributes to late detection. Another issue related to pancreatic cancer's detection is, unlike some other cancers such as breast or colorectal cancer, there are no widely recommended routine screening tests for pancreatic cancer. As such, many cases are not discovered until a person experiences symptoms or develops risk factors that lead to further investigation. Currently available methods for early detection of pancreatic cancer, such as imaging tests and blood markers, are not highly sensitive or specific, which means they may not reliably detect the disease in its early stages. As a result, tumors may go unnoticed until they are larger and more advanced. There is a critical need for diagnostic measures that will provide for early and effective detection of pancreatic cancer.

### BRIEF DESCRIPTION OF DRAWINGS

FIG. 1A is an anatomical illustration of the pancreas depicting the location and depth of the pancreas within the human body.

FIG. 1B is a schematic representation of a pancreas afflicted by a pancreatic tumor and surrounding metastasis.

FIG. 2 provides a schematic illustration of a stool sample that contains potential pancreatic cancer biomarkers (inset image showing an illustrative microscopic view of a pancreatic cancer exosome).

FIG. 3 provides a schematic illustration of an exemplary biological sample pre-conditioner according to various aspects of the disclosure.

FIG. 4 provides a schematic illustration of an exemplary biological component detector according to various aspects of the disclosure.

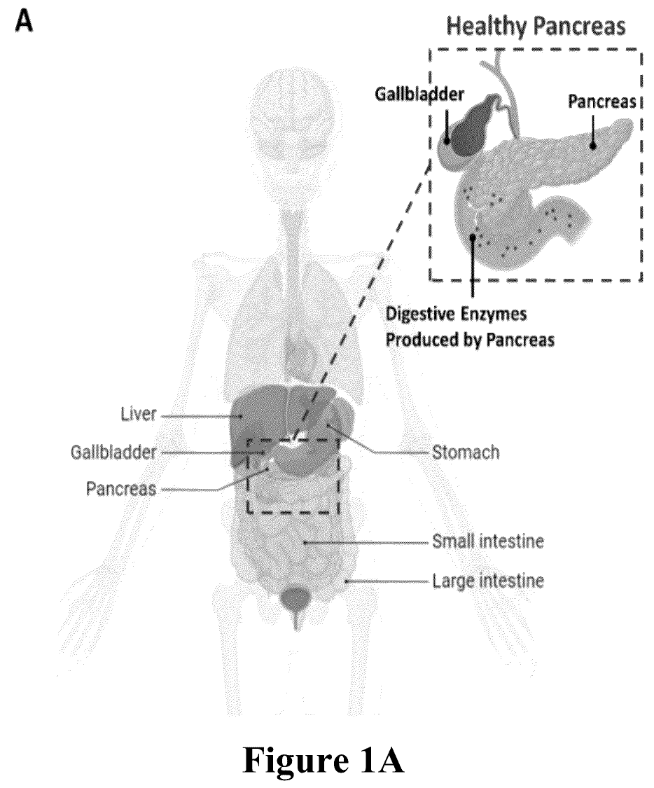

### DETAILED DESCRIPTION

Various aspects of the disclosure pertain to devices for and methods of isolating biological components from a biological sample, identifying the biological components and optionally quantifying the amount of the biological components in the biological sample. The biological samples to be isolated, identified and optionally quantified may serve as indicators of a disease state of a subject from which the biological sample originated. The devices and methods described herein may be applied to various types of biological samples. In accordance with various aspects of the disclosure, the devices and methods described herein have been found particularly useful for the isolation, identification and quantification of biological components of stool samples.

Various types of biological components in biological samples can be isolated, identified and quantified using the devices and methods described herein such as, but not limited to, nucleic acids (for example, DNA and RNA), peptides, proteins, cells, exosomes, antigens, viruses, and bacteria. In accordance with various aspects of the disclosure, the devices and methods described herein have been found particularly useful for the isolation, identification and quantification of biological components of biological samples, such as stool samples, that are indicative of various forms of cancers. More particularly, the devices and methods described herein have been found particularly useful for the isolation, identification and quantification of exosomes and antigens that are indicative of various forms of cancers, including forms of pancreatic cancers such as Exocrine Pancreatic Cancer and Endocrine Pancreatic Cancer.

Generally, devices for isolating biological components from a biological sample, identifying the biological components and quantifying the amount of the biological components in the biological sample comprise a biological sample pre-conditioner and a biological component detector. The biological sample pre-conditioner generally facilitates separation of a biological component from a bulk biological sample for subsequent identification and quantification by the biological component detector.

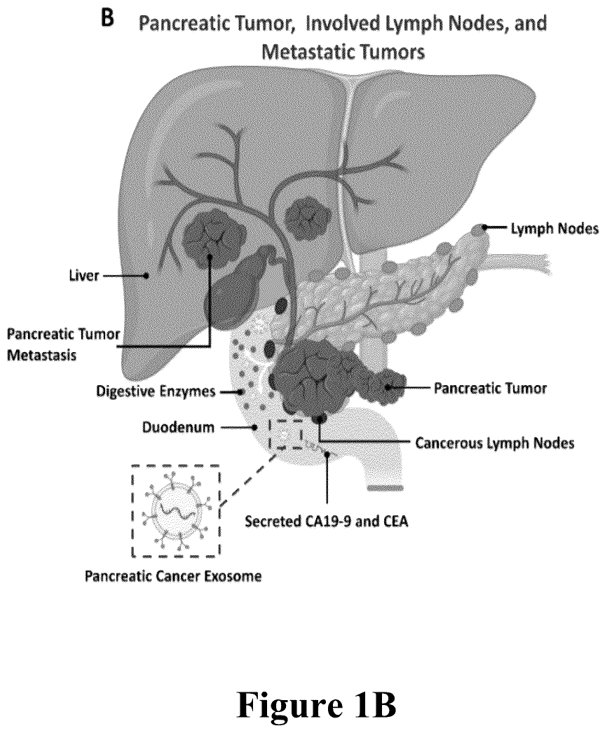

Various types of biological samples may be tested using devices according to the disclosure. In some instances, devices according to the disclosure have been found particularly for the isolation, identification and quantification of biological components in stool samples. As illustrated in FIG. 2, a stool 200 sample of a patient may comprise water 210 in an amount of from 10 to 90 wt % (for example 75 wt %) and solid waste 220 in an amount of from about 10 to 90% wt % (for example 25 wt %), based on the total weight of the stool sample 200. One of ordinary skill in the art will appreciate that various factors may result in stool samples with varying amounts of water and solid waste such as, for example, a patient's diet, hydration, use of prescription or non-prescription pharmaceuticals (for example diuretics, opioids, antidepressants, blood pressure medications, antihistamines, antacids, antibiotics, NSAIDs, chemotherapeutics, proton pump inhibitors, laxatives, and metformin), bacterial and viral infections, and so on. In addition to general solid waste generated by the body's processing of foodstuffs, a patient's stool sample may contain various biological components 230 (for example, nucleic acids (e.g., DNA and RNA), peptides, proteins, cells, exosomes, antigens, viruses, and bacteria) which serve as indicators of disease states. In FIG. 2, pancreatic cancer exosomes are illustrated as a biological component 230 to be isolated, identified and quantified by devices according to various aspects of the disclosure. Prior art methods involving taking blood samples from patients are invasive. By using stool samples, methods and devices according to the disclosure allow for less invasive testing as taking multiple stools would be far less invasive than repeatedly drawing blood samples.

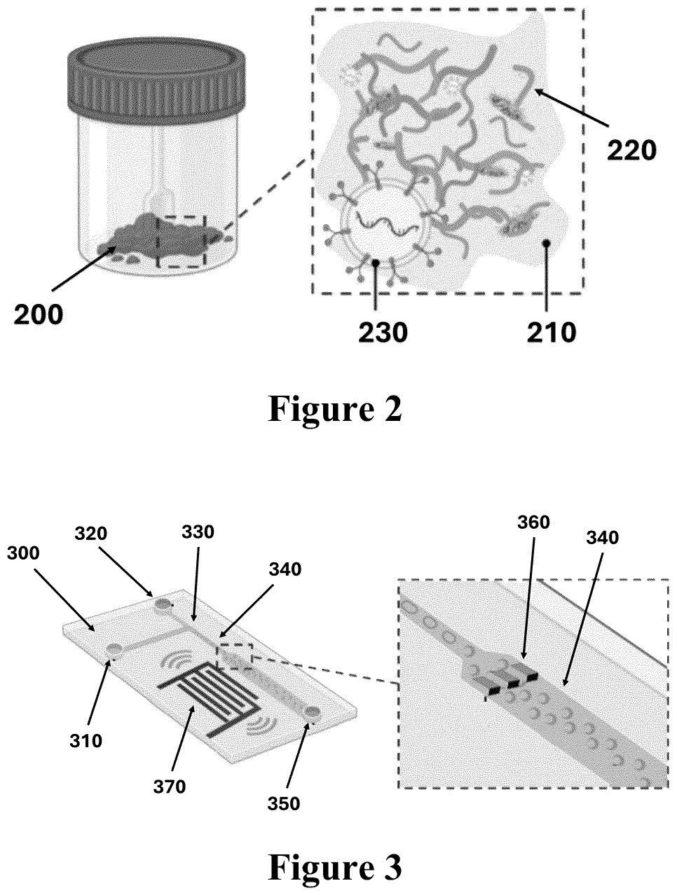

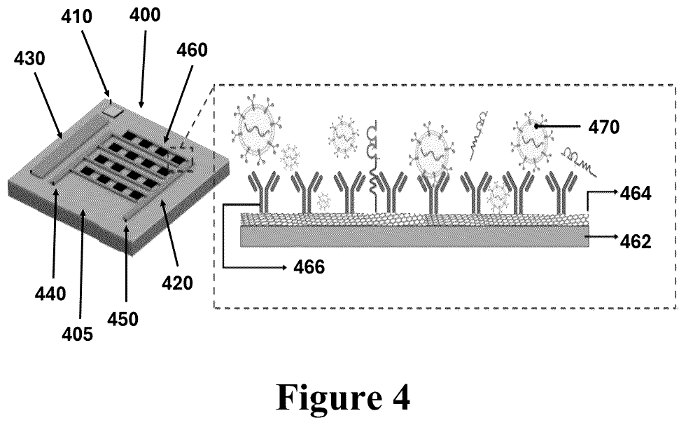

FIG. 3 is a schematic illustration of an exemplary biological sample pre-conditioner, specifically a microfluidic chip 300, according to various aspects of the disclosure. As illustrated in FIG. 3, the microfluid chip 300 includes a biological sample (for example, a stool sample) inlet 310 and a reagent solution inlet 320, each in fluid communication with a microfluidic channel 340. The reagent solution inlet 320 may be used for the addition of various reagent solutions such as pH modifiers, physiological solutions, buffers, lysing agents, isotonicity adjusters, combinations thereof, and so on. Exemplary reagent solutions include, but are not limited to PBS, HBSS, saline solutions, and cell culture media. The biological sample and reagent solution travel from their respective inlets and combine at an intersection point 330 in the microfluidic channel 340 to form a biological sample solution comprising the biological sample and the reagent solution. The microfluidic channel 340 extends from the intersection point 330 to an outlet 350 which is in fluid communication with a biological component detector as described in, for example, FIG. 4. The microfluidic channel 340 may include a plurality of flow impediments 360, which selectively reduce the flow of biological sample solution, physically breaking down the biological sample and promoting homogenization and liquification of the biological sample in the reagent solution to enhance extraction of biological components (e.g., biological components 230 such as nucleic acids (e.g., DNA and RNA), peptides, proteins, cells, exosomes, antigens, viruses, and bacteria) for subsequent detection and quantification with a biological component detector as described in, for example, FIG. 4. In some instances, microfluidic chip 300 may further comprise a micro-piezoelectric on-chip transducer 370 incorporated thereon which can agitate or shake the microfluidic chip 300 to, in conjunction with the plurality of flow impediments 360, further promote homogenization and liquification of the biological sample in the reagent solution as the biological sample solution passes through the microfluidic channel 340 from the intersection point 330 to the outlet 350.

FIG. 4 is a schematic illustration of an exemplary biological component detector 400 according to various aspects of the disclosure. As illustrated in FIG. 4, the detector 400 comprises a substrate 405, a reference electrode 410, a working electrode 420, and a counter electrode/gate 430. The working electrode 420 comprises a source 440 and a drain 450 and a plurality of conductive channels 460 electrically coupling the source 440 and the drain 450.

Each of the plurality of conductive channels 460 comprises a base substrate 462, an electrically conductive material 464, and plurality of biological species 466. Each of the plurality of conductive channels 460 extends through a portion of the substrate 405, from the top surface of the substrate 405 and terminating at a corresponding base substrate 462. In some instances, the substrate 405 and the base substrate 462 are made of the same material. The base substrate 462 comprises a layer of the electrically conductive material 464 disposed on the base substrate 462, and the plurality of biological species 466 are bound to a surface of the electrically conductive material 464. The biological species 466 have a binding affinity with one or more biological components 470 from a biological sample (see, e.g., FIG. 2).

Biological component detector 400 can be designed to operate in two distinct modes: as a field-effect transistor (FET) or as an electrochemical sensor.

In a FET mode, electrodes 430, 440, and 450 of the detector 400 function as the gate, source, and drain, respectively.

In an electrochemical mode, electrodes 440 and 450 of the detector 400, in combination, serve as the working electrode 420. In such electrochemical mode configurations, electrodes 410, 430, and 440-450 (combined as working electrode 420) correspond to the reference, counter, and working electrodes, respectively.

In electrochemical operations, the detector 400 can be used to perform various measurement techniques such as cyclic voltammetry (CV), electrochemical impedance spectroscopy (EIS), and differential pulse voltammetry (DPV), depending on the desired sensing parameters, sensitivity, and type of response. This dual-mode functionality allows for flexible application of the detector 400 depending on experimental needs.

When detector 400 is used in a FET mode, a voltage is applied to the electrodes 420 and 440 and these electrodes exhibits an initial impedance value at the applied voltage. Upon subjecting the conductive channels 460 to a liquified sample solution comprising biological components 470 (which may be obtained from the biological sample pre-conditioner 300), the biological components 470 bind with biological species 466, and the binding changes the channel conductivity and the threshold voltage. The change in threshold voltage is observed by measuring the drain current under a pre-set gate-source voltage. The degree of threshold voltage change can be correlated with the amount of biological components 470 that have bound to the biological species 466 and, by extension, the amount of biological components 470 in a given amount of a biological sample.

In some instances, detector 400 can, similar to a FET mode, be configured as a constructed bipolar junction transistor (BJT) in accordance with various aspects of the present disclosure.

The base substrate 462 can be made of various dielectric materials. Suitable dielectric materials from which the base substrate 462 can be made include, but are not limited to, polymer dielectrics (for example, polymethylmethacrylate (PMMA) and polyvinylpyrrolidone (PVP)), and SiO₂, Al₂O₃, Si₃N₄ and HfO₂. In some instances, the use of Si—SiO₂-fused Silica as the base substrate 462 is preferred. The electrically conductive material 464 can be made of various materials. In some instances, suitable materials from which the electrically conductive material 464 can be made include, but are not limited to two-dimensional (2D) materials such as graphene, black phosphorus (BP), and transition metal dichalcogenides (TMDs) of the type MX₂ where M is Mo or W, and X is S, Se or Te. In some instances, suitable materials from which the electrically conductive material 464 can be made may also include, materials that are not 2D such as, for example, carbon nanotubes, electrically conductive organic polymers, crystalline semiconducting materials in the form of thin-films (i.e., thin film transistors of TFTs) of high electron mobility materials (i.e., High electron mobility transistors or HEMTs) such as, for example, indium gallium zinc oxide (INZO), gallium nitride (GaN), aluminum gallium nitride (AlGaN), and so on. In some instances, the use of graphene as the electrically conductive material 464 is preferred. The biological species 466 can be any biological species that have a binding affinity with one or more biological components 470 that are targets for identification and quantification, such as biological species indicative of a disease state, such as a cancer or, more specifically, a pancreatic cancer. Suitable biological species 466 include, but are not limited to CEA antibodies, CA19-9 antibodies, EpCAM antibodies, CD24 antibodies, CD44 antibodies, CD62 antibodies, CD81 antibodies, CD9antibodies, and Glypican-1 (GPC1) antibodies. Such antibodies bind to various receptors on cancer exosome surfaces such as CEA, CA19-9, EpCAM, CD24, CD44, CD62, CD81, CD9 and Glypican-1 (GPC1). In some instances, the use of CEA or CA19-9 antibodies as the biological species 466 is preferred. In some instances, the normal levels of CEA in a subject range between 0 ng/mL and 3 ng/mL. In some instances, if the levels of CEA are higher than 3 ng/ml, the subject from which the biological sample is obtained may be considered high-risk and should be monitored for pancreatic cancer. In some instances, the normal range of CA 19-9 in a subject ranges between 0 and 37 U/mL. In some instances, if the levels of CA 19-9 in a subject are more than 37 U/mL, the subject from which the biological sample is obtained may be considered high-risk and should be monitored for pancreatic cancer.

In preparing the plurality of conductive channels 460, an optimal surface concentration and spacing of the biological species 466 on the electrically conductive material 464 is desirable to maximize the detection of biological components 470. If the surface concentration of the biological species 466 on the electrically conductive material 464 is too high, steric hindrance can reduce binding efficiency. If the surface concentration of the biological species 466 on the electrically conductive material 464 is too low, detection sensitivity decreases due to fewer binding events. In this regard, the inventors have determined, spacing between adjacent biological 466 species should generally be in the 10-30 nm range, with a biological species 466 surface coverage on the electrically conductive material 464 being generally within 1-10 pmol/cm². In some instances, the use of spacers such as PEG can help tune spacing and reduce non-specific interactions and we can mention here as we have tested before.

In some instances, such as when carcinoembryonic antigen (CEA) antibody is used as the biological species 466 on the electrically conductive material 464, it has been found that immobilization of a sufficient amount of CEA antibody to provide a 1-10 pmol/cm² surface concentration on the electrically conductive material 464 can be achieved by treating the electrically conductive material 464 with a solution having a CEA antibody concentration of approximately 20 μg/mL.

In some instances, such as when carbohydrate antigen 19-9 (CA19-9) antibody is used as the biological species 466 on the electrically conductive material 464, it has been found that immobilization of a sufficient amount of CA19-9 antibody to provide a 1-10 pmol/cm² surface concentration on the electrically conductive material 464 can be achieved by treating the electrically conductive material 464 with a solution having a CA19-9 antibody concentration of approximately 10 μg/mL.

In some instances, such as when EpCAM antibody is used as the biological species 466 on the electrically conductive material 464, it has been found that immobilization of a sufficient amount of EpCAM antibody to provide a 1-10 pmol/cm² surface concentration on the electrically conductive material 464 can be achieved by treating the electrically conductive material 464 with a solution having an EpCAM antibody concentration of approximately 25 μg/mL.

In some instances, such as when CD24 antibody is used as the biological species 466 on the electrically conductive material 464, it has been found that immobilization of a sufficient amount of CD24 antibody to provide a 1-10 pmol/cm² surface concentration on the electrically conductive material 464 can be achieved by treating the electrically conductive material 464 with a solution having a CD24 antibody concentration of approximately 0.2 mg/mL.

In some instances, such as when CD44 antibody is used as the biological species 466 on the electrically conductive material 464, it has been found that immobilization of a sufficient amount of CD44 antibody to provide a 1-10 pmol/cm² surface concentration on the electrically conductive material 464 can be achieved by treating the electrically conductive material 464 with a solution having a CD44 antibody concentration of approximately 20 μg/mL.

In some instances, such as when CD62P (P-Selectin) antibody is used as the biological species 466 on the electrically conductive material 464, it has been found that immobilization of a sufficient amount of CD62P antibody to provide a 1-10 pmol/cm² surface concentration on the electrically conductive material 464 can be achieved by treating the electrically conductive material 464 with a solution having a CD62P antibody concentration of approximately 0.5 mg/mL.

In some instances, such as when CD81 antibody is used as the biological species 466 on the electrically conductive material 464, it has been found that immobilization of a sufficient amount of CD81 antibody to provide a 1-10 pmol/cm² surface concentration on the electrically conductive material 464 can be achieved by treating the electrically conductive material 464 with a solution having a CD81 antibody concentration of approximately 1 μg/μL.

In some instances, such as when CD9 antibody is used as the biological species 466 on the electrically conductive material 464, it has been found that immobilization of a sufficient amount of CD9 antibody to provide a 1-10 pmol/cm² surface concentration on the electrically conductive material 464 can be achieved by treating the electrically conductive material 464 with a solution having a CD9 antibody concentration of approximately 5 μL/test.

In some instances, such as when Glypican-1 (GPC1) antibody is used as the biological species 466 on the electrically conductive material 464, it has been found that immobilization of a sufficient amount of GPC1 antibody to provide a 1-10 pmol/cm² surface concentration on the electrically conductive material 464 can be achieved by treating the electrically conductive material 464 with a solution having a GPC1 antibody concentration of approximately 7.5 μg/mL.

The biological species 466 surface concentrations may be modified within an optimized ranges to ensure minimal steric hindrance while maximizing specific binding interactions, particularly when combined with surface modifiers like PEG to enhance orientation and reduce nonspecific interactions.

### EXAMPLES

Conventional methods currently used for detecting CA19-9 and CEA in blood samples can be adapted for use with stool samples. Enzyme-linked immunosorbent assay (ELISA) is one such method that can be employed to quantify the levels of these markers in stool. Another innovative approach involves the development of biosensors, such as electrochemical sensors, for the detection of CA19-9 and CEA in stool samples. Biosensors offer advantages such as high sensitivity and specificity, rapid results, and potentially lower costs. These sensors can be fabricated with specific antibodies or molecular probes that bind to the markers, generating a measurable electrical signal. Various aspects of the disclosure relate to the realization of sensor arrays with micron-size field-effect transistors (FETs) for the detection of pancreatic cancer exosomes. In some instances, both silicon (Si)-based and graphene-based FETs (GFETs) can enable the sensing performance and are manufacturable. In some instances, GFETs are preferably surface-functionalized with CEA or CA19-9 antibodies, to bind to CEA or CA19-9 biomarkers on the surface of an exosome, respectively, due to higher sensitivity in charge detection using liquid-gate FET configuration. When the target biomarker binds to its corresponding antibody on the GFET, the charge of the biomarker changes the FET channel conductivity and changes the threshold voltage. The change in threshold voltage is observed by measuring the drain current under a properly set gate-source voltage. A similar mechanism can be used for electrochemical biosensors based on cyclic voltammetry (CV), electrochemical impedance spectroscopy (EIS), and differential pulse voltammetry (DPV).

To reduce non-specific binding (Frutiger A, Tanno A, Hwu S, Tiefenauer R F, Vörös J, Nakatsuka N. Nonspecific Binding—Fundamental Concepts and Consequences for Biosensing Applications. Chem Rev 2021; 121:8095-8160. https://doi.org/10.1021/acs.chemrev.1c00044) and improve the sensitivity of the FET-based biosensor, a layer of silicon oxide (SiO₂) can be applied to all metal connectors, including the gold gate, source, and drain ports, as well as the electrodes. Additionally, to minimize non-specific binding on the GFET channel and increase the Debye length (Chu C H, Sarangadharan I, Regmi A, Chen Y W, Hsu C P, Chang W H, et al. Beyond the Debye length in high ionic strength solution: Direct protein detection with field-effect transistors (FETs) in human serum. Sci Rep 2017; 7:1-15. https://doi.org/10.1038/s41598-017-05426-6), a low concentration of poly (ethylene glycol) (PEG) can be introduced onto the channel surface. The Debye length is a critical parameter to consider because the sensitivity of electronic biosensors can be restricted by the shielding of electric fields caused by mobile ions found in biological samples. That phenomenon leads to the electric “double layer” effect, which has posed a significant challenge in electronic detection platforms. The Debye length represents the spatial scale of this shielding and is typically less than 1 nm in physiological conditions. In contrast, antibodies and aptamers have dimensions measured in several nanometers. To address those challenges, a high-frequency carrier signal (up to tens of megahertz) can be modulated with a lower-frequency signal and injected through the source terminal of the FET-based biosensor. That action induces oscillations in dipoles at the gate terminal, resulting in the generation of a detection signal. This approach combines straightforward low-frequency readout instrumentation with the behavior of high-frequency screening. At even higher frequencies (in the gigahertz range), the double layer formation does not occur, allowing the electric field to penetrate the electrochemical cell. Consequently, the cell behaves like a single capacitor, with the electrolyte serving as the dielectric material. By measuring its capacitance and dielectric properties, we can quantify the cell's charge.

FIGS. 3 and 4, as discussed above, illustrate an exemplary device for liquefying stool samples and detecting pancreatic cancer indicators through multimodal sensing. FIG. 3 schematically illustrates a microfluidic chip, according to various aspects of the disclosure, engineered to liquefy a stool sample by, for example, employing a mixing mechanism with a physiological solution, such as phosphate-buffered saline, and utilizing ultrasonic transducers (e.g., micro-piezoelectric on-chip transducer 370) to transform the laminar flow within the chip into an agitated flow, aiding in the liquefaction process. The ultrasonic transducers, fabricated using nanostructures like zinc oxide nanowires, will be situated at the end of the channel where a filter will segregate large debris, preparing the sample for interaction with the biosensor. FIG. 4 schematically illustrates a FET-based biosensor according to various aspects of the disclosure, designed for multimodal sensing to concurrently detect and quantify target molecular markers, such as CA19-9 and CEA. GFET electrodes, functioning as gate, drain, and source, operates as an electrochemical sensor, serving alternately as the counter electrode (gate), working electrode (source and drain), and reference electrode. By immobilizing antibodies specific to EpCAM, CD24, CD44, CD63, CD81, CD9 and Glypican-1 (GPC1) on the conductive channels (460), the biosensor array will also be capable of detecting pancreatic cancer exosomes. In such a configuration, a liquid biological sample is applied on top of all the conductive channels in such a way that it bridges the gate, source, and drain electrodes. This allows the electrolyte to act as the gating medium, enabling the gate voltage to modulate the channel conductivity through the liquid interface. The sample itself serves as both the biological medium and the electrical bridge, making this setup ideal for direct and even label-free detection in complex fluids.

In some instances, one point of novelty of methods according to the disclosure lie in the use of stool samples for monitoring CA19-9 and CEA levels, a significant departure from traditional blood sample analysis. Traditionally, blood tests have been the primary mode for detecting these biomarkers, but they can be invasive and uncomfortable. The new stool sample-based analysis methodologies offer a non-invasive and more patient-friendly alternative. Furthermore, stool sample-based analysis methodologies according to various aspects of the disclosure may allow for detecting changes in biomarker levels earlier than blood tests, as those markers could appear in stool samples sooner. This innovation may lead to earlier detection of pancreatic cancer, enhancing treatment outcomes and patient prognosis. This shift to stool analysis represents a significant advancement in the field of cancer diagnostics.

The advantages of the methods and devices described herein are multifaceted. They offer a non-invasive alternative to blood tests, which is less discomforting and more accessible for regular monitoring. Such approaches are particularly beneficial for screening family members with a history of pancreatic cancer, as it allows for easy, regular check-ups without the need for medical professionals. Unlike other methods that require trained operators, this stool analysis can be utilized by individuals with a normal level of education, making it a practical option for home-based screening. This ease of use, combined with the potential for earlier detection and monitoring, marks a significant improvement over existing methods, which often face limitations in early cancer detection.

Commercial applications of stool analysis methods, according to various aspects of the disclosure, for early pancreatic cancer detection extend beyond the area of medical diagnostics. The economic potential for stool analysis methods according to the disclosure is significant due to its role in facilitating early detection, which can lead to timely and more effective treatment, potentially improving patient outcomes. Stool analysis methods according to the disclosure also find direct application in routine medical diagnostics, which is particularly beneficial for high-risk groups like families with a history of pancreatic cancer. Furthermore, the simplicity and non-invasive nature of stool analysis methods according to the disclosure position then well for use in continuous health monitoring and long-term research studies, broadening their impact across various sectors of healthcare.

What is claimed is:

1. A method for evaluating the presence of a cancer within a subject, the method comprising:
obtaining a stool sample from the subject;
identifying a biological component in the stool sample, wherein the identity of the biological component is indicative of the presence or absence of a cancer.

2. The method of claim 1, further comprising quantifying the amount of the biological component in the stool sample, wherein the quantity of the biological component is indicative of the presence or absence of a cancer.

3. The method of claim 1, wherein the cancer is pancreatic cancer.

4. The method of claim 1, wherein the biological component is an exosome.

5. The method claim 1, wherein the identity of the biological component is determined based on its binding affinity with a binding agent located on an electrochemical device.

6. The method of claim 5, wherein the binding agent is an antibody.

7. The method of claim 6, wherein the antibody is CEA antibody, CA19-9 antibody, EpCAM antibody, CD24 antibody, CD44 antibody, CD62 antibody, CD81 antibody, CD9 antibody, or any combination thereof.

8. The method of claim 5, wherein the electrochemical device is configured as a field-effect transistor (FET).

9. The method of claim 8, wherein the FET is a silicon-based FET or a graphene-based FET.

10. The method of claim 5, wherein the electrochemical device is configured for cyclic voltammetry (CV) measurements, electrochemical impedance spectroscopy (EIS) measurements, or differential pulse voltammetry (DPV) measurements.

11. The method of claim 1, further comprising homogenizing and/or liquefying the stool sample prior to identifying the biological component.

12. A device for evaluating the presence of cancer within a subject, the device comprising:
a biological sample pre-conditioner, wherein the biological sample pre-conditioner is configured to prepare a biological sample for quantification of a biological component in the biological sample; and
a biological component detector, wherein the biological component detector is configured to identify the biological component in the biological sample;
wherein the identity of the biological component in the biological sample is indicative of the presence or absence of a cancer.

13. The device of claim 12, wherein the biological component detector is further configured provide data indicative of the quantity of the biological component in the biological sample;
wherein the quantity of the biological component in the biological sample is indicative of the presence or absence of a cancer.

14. The device of claim 12, wherein the biological sample is a stool sample.

15. The device of claim 12, wherein the biological sample pre-conditioner comprises:
a biological sample inlet;
a reagent solution inlet;
a microfluidic channel in fluidic communication with the biological sample inlet and the reagent solution inlet, and
a biological solution outlet in fluidic communication with the biological component detector, wherein
the biological sample from the biological sample inlet and reagent solution from the reagent solution inlet combine in the microfluidic channel to form a biological sample solution from transmission of the biological sample solution from the biological solution outlet to the biological component detector.

16. The device of claim 12, wherein the biological sample detector comprises:
a reference electrode; and
a working electrode comprising:
a source;
a drain; and
a plurality of conductive channels electrically coupling the source and the drain, each of the plurality of conductive channels comprising biological species bound on surfaces thereof, the biological species having a binding affinity with the biological component.

17. The device of claim 16, wherein the biological component is an exosome or antigen of a cancer.

18. The device of claim 16, wherein the biological component is an exosome or antigen of a pancreatic cancer.

19. The device of claim 18, wherein the biological species is an antibody.

20. The device of claim 19, wherein the antibody is CEA antibody, CA19-9 antibody, EpCAM antibody, CD24 antibody, CD44 antibody, CD62 antibody, CD81 antibody, CD9 antibody, or any combination thereof.
